(19) United States
(12) Patent Application Publication
(10) Pub. No.: US 2008/0319498 A1
(43) Pub. Date: Dec. 25, 2008

(54) Apparatus and Method for Detecting Diastolic Heart Failure

(76) Inventor: Sven-Erik Hedberg, Kungsangen (SE)

Correspondence Address: SCHIFF HARDIN, LLP;PATENT DEPARTMENT, 6600 SEARS TOWER, CHICAGO, IL, 60606-6473, US

(21) Appl. No.: 11/658,981

(22) PCT Filed: Aug. 31, 2004
(86) PCT No.: PCT/SE04/01247

§ 371 Date: Jan. 29, 2007

(51) Int. Cl. A61N 1/365; A61B 5/02

(52) U.S. Cl. 607/17; 600/508

(57) ABSTRACT

In a method and implantable medical apparatus for detecting diastolic heart failure (DHF), and a pacemaker embodying such an apparatus, movement of the valve plane of the heart is measured and analyzed to identify a slowing of the movement of the valve plane as an indication of a DHF state of the heart. A signal indicative of this DHF state is emitted and, in the pacemaker, is used to control the administration of a pacing pulse therapy to the heart.

### TECHNICAL FIELD

The present invention relates to an implantable medical apparatus and a method for detecting diastolic heart failure, DHF, and a pacemaker comprising said apparatus.

### BACKGROUND OF THE INVENTION

There is a growing recognition that congestive heart failure caused by a predominant abnormality in the diastolic function, i.e. diastolic heart failure, DHF, is both common and causes significant morbidity and mortality. Therefore early detection of DHF is important. Patients do not, however, seem to have symptoms at an early stage. In addition it has been hard to separate diastolic and systolic heart failure, and they may also exist simultaneously.

DHF is characterized by a slowing down of the recoil effect during early diastole, i.e. during the isovolumic ventricular relaxation and the rapid left ventricular filling phase, before the atrial contraction. This has been observed by measuring the velocity of the mitral annulus. According to an article by Margaret M. Redfield et. al., “Burden of Systolic and Diastolic Ventricular Dysfunction in the Community, JAMA, Vol. 289, No. 2, p. 194-202, Jan. 8, 2003, the velocity of the mitral annulus motion reflects the state of DHF. The article shows that the velocity of the mitral annulus motion decreases when the diastolic function gets more deteriorated.

However, the measurement of heart tissue has already been used for other purposes. For example, U.S. Pat. No. 5,480,412 A discloses a processing system and method for deriving an improved hemodynamic indicator from cardiac wall acceleration signals. The cardiac wall acceleration signals are provided by a cardiac wall sensor that responds to cardiac mechanical activity. The cardiac wall acceleration signals are integrated over time to derive cardiac wall velocity signals, which are further integrated over time to derive cardiac wall displacement signals. The cardiac wall displacement signals correlate to known hemodynamic indicators. An implantable cardiac stimulating device using cardiac wall displacement signals to detect and discriminate cardiac arrhythmias is also described. Further, U.S. Pat. No. 5,628,777 A discloses an implantable lead comprising an accelerometer-based cardiac wall motion sensor. Said sensor transduces accelerations of cardiac tissue to provide electrical signals indicative of cardiac wall motion to an implantable cardiac stimulation device. Said device uses said electrical signals to detect and discriminate among potentially malignant cardiac arrhythmias. Furthermore, U.S. Pat. No. 6,009,349 A discloses a processing system for an implantable cardiac device, said device having cardiac wall accelerator sensors for providing a cardiac wall accelerator signal as a function of cardiac wall contractile motion, the sensors being positioned in the right atrium and right ventricle of a patent's heart.

### THE OBJECT OF THE INVENTION

The object of the present invention is to utilize above mentioned knowledge to propose a technique for detecting DHF, preferably at an early stage when the patient still does not seem to have any symptoms, based on the movement of the mitral annulus.

### SUMMARY OF THE INVENTION

The above-mentioned object is achieved by an apparatus, a pacemaker, and a method of the kind mentioned in the introductory portion of the description and having the characterizing features of claims 1, 16 and 17, respectively. It has been shown that the movement of the valve plane of the heart is comparable to the movement of the mitral annulus, so by the apparatus, pacemaker, and method of the present invention is an efficient technique for detecting DHF provided, also for detecting DHF at an early stage when the patient still does not seem to have any symptoms.

According to advantageous embodiments of the apparatus according to the present invention, the analysing means comprise a comparison means for comparing the measured movement of the valve plane with predetermined reference values, and the measuring means is arranged to measure the movement of the valve plane during early diastole, before the atrial contraction.

According to another advantageous embodiment of the apparatus according to the present invention, the apparatus comprises first detection means for detecting the T-wave, and the measuring means is arranged to measure the movement of the valve plane in the vicinity of the T-wave.

According to a further advantageous embodiment of the apparatus according to the present invention, the apparatus comprises second detection means for detecting the QRS complex, and the measuring means is arranged to measure the movement of the valve plane during a time window starting just after the QRS complex and ending before the atrial contraction.

According to an advantageous embodiment of the apparatus according to the present invention, the apparatus comprises activity measuring means for measuring the condition of the patient, and the measuring means is arranged to measure the movement of the valve plane when the activity measuring means indicates resting conditions of the patient.

According to a further advantageous embodiment of the apparatus according to the present invention, the measuring means is arranged to measure movement of the valve plane by measuring the velocity or the acceleration of the valve plane.

According to other advantageous embodiments of the apparatus according to the present invention, the measuring means comprises an accelerometer arranged to be placed on or close to the valve plane, and the apparatus comprises calculating means for calculating the velocity of the valve plane by summing-up or integrating the measured acceleration values.

According to still other advantageous embodiments of the apparatus according to the present invention, the measuring means is arranged to measure the pressure from the inner walls of the coronary sinus, the great cardiac vein or a coronary vein, said pressure correlating with the movement of the valve plane, and the measuring means arranged to measure said pressure comprises a pressure sensor with a circumferential sensitivity arranged to be placed in the coronary sinus, the great cardiac vein or in a coronary vein.

According to yet other advantageous embodiments of the apparatus according to the present invention, the analysing means are arranged to find the peak value from measured values from one heart interval, the apparatus comprises an averaging means for forming an average value of peak values from measured values from several heart intervals, and the apparatus comprises storing means for storing measured values together with the time of occurrence, for later analysis.

According to advantageous embodiments of the method according to the present invention, the measured movement of the valve plane is compared with predetermined reference values, and the movement of the valve plane is measured during early diastole, before the atrial contraction.

According to a further advantageous embodiment of the method according to the present invention, the T-wave is detected, and the movement of the valve plane is measured in the vicinity of the T-wave.

According to another advantageous embodiment of the method according to the present invention, the QRS complex is detected, and the movement of the valve plane is measured during a time window starting just after the QRS complex and ending before the atrial contraction.

According to other advantageous embodiments of the method according to the present invention, the condition of the patient is measured, and the movement of the valve plane is measured, by measuring the velocity or the acceleration of the valve plane, during resting conditions of the patient, and if the acceleration of the valve plane is measured, the velocity of the valve plane is calculated from the acceleration by summing-up or integrating the measured acceleration values, e.g.

According to yet another advantageous embodiment of the method according to the present invention, the pressure is measured from the inner walls of the coronary sinus, the great cardiac vein or a coronary vein, said pressure correlating with the movement of the valve plane.

According to still other advantageous embodiments of the method according to the present invention, the method comprises the special measures of finding the peak value from measured values from one heart interval, forming an average value of peak values from measured values from several heart intervals, and storing measured values together with the time of occurrence, for later analysis.

### BRIEF DESCRIPTION OF THE DRAWINGS

The present invention will now be described, for exemplary purposes, in more detail by way of embodiments and with reference to the enclosed drawings, in which:

FIG. 1 shows diagrams from the mentioned article by Margaret M. Redfield et. al., showing the velocity of the mitral annulus during early diastole in relation to the degree of DHF,

FIG. 2 shows a schematic block diagram of an embodiment of a pacemaker according to the present invention, and

FIG. 3 shows a flow chart illustrating an embodiment of the method according to the present invention.

### DETAILED DESCRIPTION OF PREFERRED EMBODIMENTS

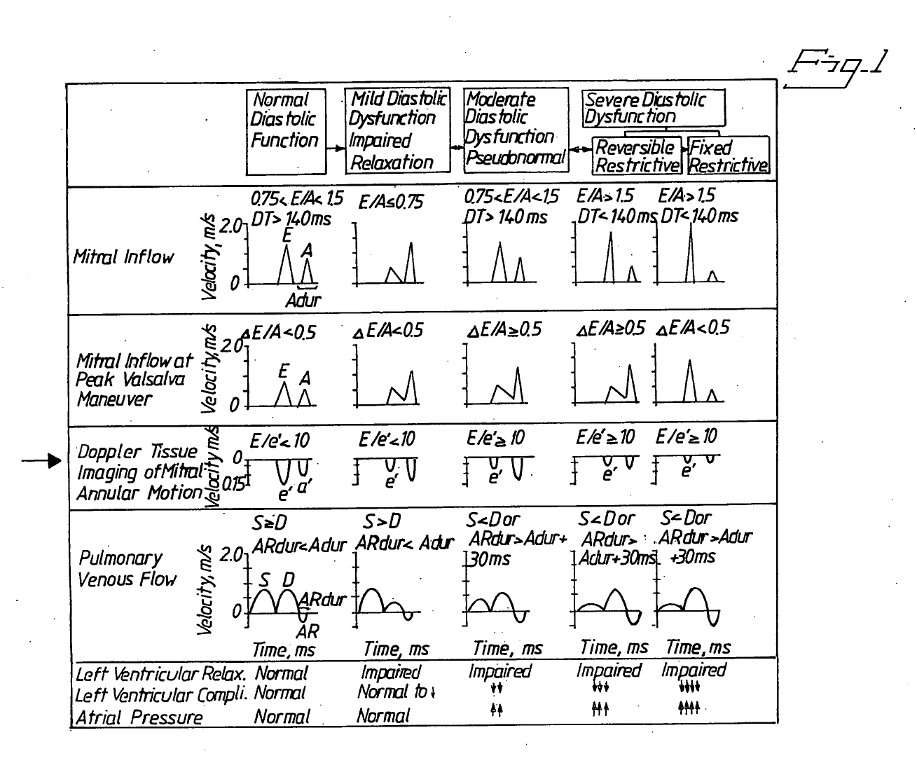

In the diagrams for Doppler Tissue Imaging of Mitral Annular Motion of FIG. 1, the curve e′ shows the velocity of mitral annulus motion during early diastole. It is clear from the diagrams that the velocity of the mitral annulus motion decreases when the diastolic dysfunction gets more severe.

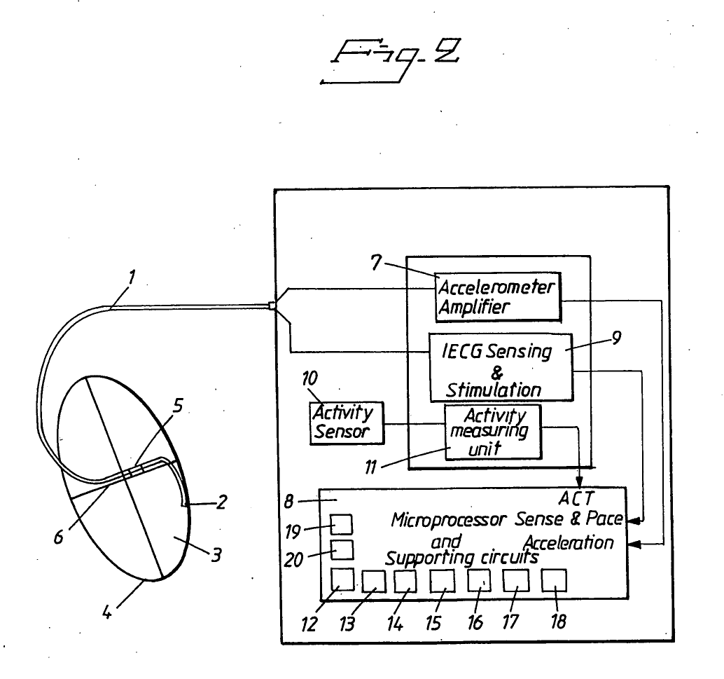

FIG. 2 shows an embodiment of a pacemaker comprising an apparatus according to the present invention. The pacemaker is adapted for left ventricular pacing only, and a left ventricular lead 1 of the pacemaker is with its electrode 2 connected to the left ventricle 3 of a patent's heart 4. Integrated with the lead 1 is an accelerometer 5 which is placed in the valve plane 6 of the patent's heart 4, but can also be placed close to the valve plane 6 in the lower part of the right atrium, or inside one of the ventricles. The accelerometer 5 is arranged to measure the acceleration of the valve plane 6 and is via the lead 1 connected to an accelerometer amplifier 7 arranged to amplify the acceleration signals, which in turn is connected to microprocessor and supporting circuits 8 of the pacemaker. The electrode 2 of the lead 1 is connected to IECG sensing and stimulation means 9 which in turn is connected to the microprocessor and supporting circuits 8.

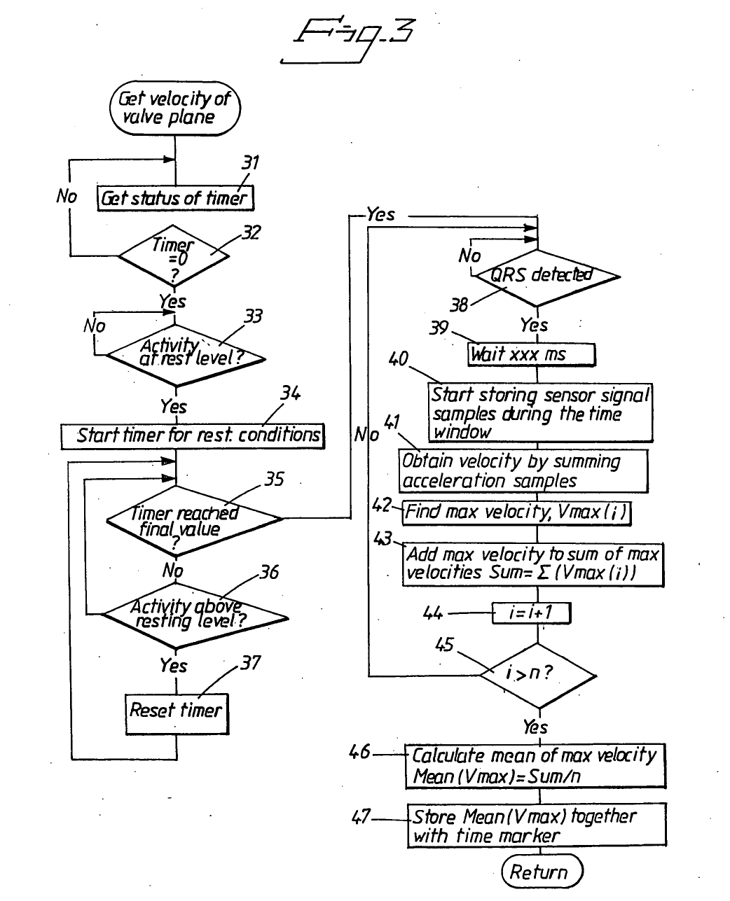

The DHF is a slow process. Therefore, the points in time for measuring the acceleration of the valve plane 6 is preferably applied to occasions when the patient is only making small movements and the signal interference is low, e.g. during sleep. In order to identify such moments the pacemaker comprises an activity sensor 10 connected to an activity measuring unit 11 for measuring the condition of the patient, which unit 11 in turn is connected to the microprocessor and supporting circuits 8. The microprocessor and supporting circuits 8 provide a timer 12 for starting the process of measuring the movement of the valve plane 6, the function of which is described in connection to FIG. 3.

The diastolic phase of interest is during the isovolumic ventricular relaxation and the rapid left ventricular filling phase, before the atrial contraction, thus the microprocessor and supporting circuits 8 provide detection means 13 for detecting the QRS complex from the signals captured by the electrode 2. The occurrence of peak velocity of the valve plane 6 takes place only a small varying time delay after the QRS complex. The microprocessor and supporting circuits 8 are arranged to lay down a time window, with a width of about 100 ms, enough to cover the valve plane motion during the relaxation phase, which starts just after the QRS complex and ends before the atrial contraction, and the accelerometer 5 is arranged to measure the acceleration of the valve plane 6 during said time window. The microprocessor and supporting circuits 8 provide calculating means 14 for calculating the velocity of the valve plane 6 by summing-up or integrating the measured acceleration values during said time window. The microprocessor and supporting circuits 8 also provide analysing means 15 for finding the peak value from measured values from one heart interval. Further, the microprocessor and supporting circuits 8 provide storing means 16 for storing measured values together with the time of occurrence, for later analysis. Thus, the development of valve plane movement values over time can be obtained. Additionally, the microprocessor and supporting circuits 8 provide averaging means 17 for forming an average value of peak values from measured values from several heart intervals. The analysing means 15 are arranged to analyse the measurement of the movement of the valve plane 6, and comprise a determining means 18 for determining a slowdown of the movement of the valve plane 6 for indicating a DHF state of the heart 4 of a patient from the determined slowdown. Further, the analysing means 15 comprise a comparison means 19 for comparing the measured movement of the valve plane 6 with pre-determined reference values. The comparison with reference values supports the indication of a DHF state. Finally, the microprocessor and supporting circuits 8 provide control means 20 for optimising pacing therapy depending on the result of the analysis of the measured movement of the valve plane 6.

If the implantation of the pacemaker occurs at a point in time when no essential DHF is at hand, the peak velocity obtained will be the basis for evaluating the degree of DHF. The pacemaker can also measure absolute peak velocity if the processed accelerometer signal is calibrated. This can be done by comparing the peak velocity found by the pacemaker with the peak velocity found by ultrasonic equipment suitable for such measurements. It is enough to measure and calibrate one peak velocity, since the accelerometer will show zero signal at zero velocity.

FIG. 3 shows a flow chart illustrating an embodiment of the method according to the present invention. Since velocity measurement of the valve plane should be carried out during resting condition of the patient, e.g. during sleep, the pacemaker comprises a timer which starts the process of measuring the movement of the valve plane. First the status of the timer is checked, at 31. If the timer has counted down to zero, at 32, the next step is to wait for a resting period, at 33, long enough to ensure resting condition of the patient. When a resting period occurs, the timer starts, at 34. If the activity of the patient is not above resting level, at 36 and when the timer reaches its final value, at 35, the detection of the QRS complex starts, at 38. If the activity of the patient is above resting level, at 36, the timer is reset, at 37.

When the QRS complex is detected, a delay is laid out, at 39, at the end of which the time window is opened, and the storing of accelerator signal samples during the time window starts, at 40. The acceleration samples from the accelerator are integrated or summed up, at 41, to obtain the velocity, and the peak velocity is found during said time window, at 42, and added to a sum of peak velocities, at 43. The steps 38 to 44 are repeated for n heart intervals, and when peak velocities from n heart intervals have been collected, at 45, and added to the sum of peak velocities, at 43, an average value of the peak velocities from the n heart intervals is formed, at 46, by dividing the sum of peak velocities by the number of heart intervals, n. The number n may be in the order of 10, but it is not a critical number. The average value of the peak velocities is stored together with the time of occurrence, at 47, for analysis in order to detect a DHF state of the heart of a patient. Said analysis comprises the step of determining a slowdown of the movement of the valve plane for indicating a DHF state of the heart of a patient from the determined slowdown.

I claim as my invention:

1-29. (canceled)

30. An implantable medical apparatus for detecting diastolic heart failure of the heart of a patient, comprising:
a movement of the valve plane of the heart that emits an electrical signal representing a measure of movement of the valve plane of a heart;
a movement analyzer, supplied with said electrical signal, that analyzes the movement of the valve plane represented by said electrical signal; and
said movement analyzer comprising a determination unit that identifies slowing of said movement of said valve plane and that, upon identifying said slowing, emits a signal indicating a DHF state of the heart of the patient.

31. An apparatus as claimed in claim 30 wherein said movement analyzer comprises a comparator that compares said measurement of said movement of said valve plane with at least one predetermined reference value.

32. An apparatus as claimed in claim 30 wherein said movement analyzer identifies a time range comprising early diastole, before an atrial contraction, of said heart and analyzes said measurement of said movement of said valve plane in said time range.

33. An apparatus as claimed in claim 32 wherein said movement analyzer comprises a detector that detects a T-wave of the heart, and wherein said movement analyzer analyzes said measurement of said movement of the valve plane at a time substantially coinciding with said T-wave.

34. An apparatus as claimed in claim 30 comprising an activity sensor that detects an activity condition of the patient and that emits a further electrical signal to said movement analyzer representing said activity condition, and wherein said movement analyzer analyzes said measurement of said movement of the valve plane when said further electrical signal indicates a resting condition of the patient.

35. An apparatus as claimed in claim 30 wherein said movement of said valve plane exhibits a velocity, and wherein said movement measuring unit measures said velocity.

36. An apparatus as claimed in claim 30 wherein said movement of said valve plane exhibits an acceleration, and wherein said movement measurement unit measures said acceleration.

37. An apparatus as claimed in claim 36 wherein said movement measurement unit is an accelerometer configured for placement on or close to said valve plane.

38. An apparatus as claimed in claim 37 wherein said movement analyzer comprises a calculating unit that calculates a velocity of said valve plane by summing or integrating respective values representing said acceleration measured by said movement measuring unit.

39. An apparatus as claimed in claim 30 wherein said movement measurement unit measures a pressure correlating movement of said valve plane, at a location selected from the group consisting of an inner wall of the coronary sinus, an inner wall of the great cardiac vein, and an inner wall of a coronary vein.

40. An apparatus as claimed in claim 39 wherein said movement measurement unit is a pressure sensor having a circumferential sensitivity configured for placement at said location.

41. An apparatus as claimed in claim 30 wherein said electrical signal representing said movement of the valve plane exhibits a peak value within each cardiac cycle, and wherein said movement analyzer analyzes said movement of said valve plane by identifying said peak value in one cardiac cycle.

42. An apparatus as claimed in claim 30 wherein said electrical signal representing said movement of the valve plane exhibits a peak value within each cardiac cycle, and wherein said movement analyzer analyzes said movement of said valve plane by forming an average value of respective peak values in a plurality of cardiac cycles.

43. An apparatus as claimed in claim 30 comprising a storage unit that stores said electrical signal together with a time at which said electrical signal was measured, said storage unit being accessible for subsequent retrieval of said electrical signal and said time stored therein.

44. An implantable pacemaker comprising:
a pulse generator having at least one electrode connected thereto for delivering a cardiac pacing therapy, comprising pacing pulses, to a heart of a patient;
an apparatus for detecting diastolic heart failure (DHF) of the heart, comprising a movement of the valve plane of the heart that emits an electrical signal representing a measure of movement of the valve plane of a heart, a movement analyzer, supplied with said electrical signal, that analyzes the movement of the valve plane represented by said electrical signal, and said movement analyzer comprising a determination unit that identifies slowing of said movement of said valve plane and that, upon identifying said slowing, emits a signal indicating a DHF state of the heart of the patient; and
a control unit that controls said pulse generator dependent on said signal indicating said DHF state.

45. A method for detecting diastolic heart failure (DHF) of the heart of a patient, comprising the steps of:
measuring movement of a valve plane of the heart and generating an electrical signal representing said movement;
automatically electronically analyzing said movement represented by said electrical signal by identifying a slowing of said movement of the valve plane; and
emitting a further electrical signal, indicating a DHF state of the heart of the patient, upon identification of said slowing.

46. A method as claimed in claim 45 comprising identifying said slowing by comparing said electrical signal representing said movement with at least one predetermined referenced value.

47. A method as claimed in claim 46 comprising measuring said movement of the valve plane during early diastole of the heart, before an atrial contraction.

48. A method as claimed in claim 47 comprising detecting a T-wave of the heart, and measuring said movement of the valve plane at a time substantially coincided with said T-wave.

49. A method as claimed in claim 48 comprising detecting a QRS complex of the heart, and measuring said movement of the valve plane during a time window starting immediately after said QRS complex and ending before a next atrial contraction.

50. A method as claimed in claim 47 comprising detecting an activity condition of the patient, and measuring said movement of the valve plane during a resting condition of the patient.

51. A method as claimed in claim 47 wherein said movement of said valve plane exhibits a velocity, and comprising measuring said movement of the valve plane by measuring said velocity.

52. A method as claimed in claim 47 wherein said movement of said valve plane exhibits a acceleration, and comprising measuring said movement of the valve plane by measuring said acceleration.

53. A method as claimed in claim 52 comprising calculating a velocity of said movement of the valve plane by summing or integrating a plurality of values respectively representing said acceleration.

54. A method as claimed in claim 47 comprising measuring said movement of the valve plane by measuring a pressure correlating with said movement of the valve plane at a location selected from the group consisting of an inner wall of the coronary sinus, an inner wall of the great cardiac vein, and an inner wall of a coronary vein.

55. A method as claimed in claim 47 wherein said electrical signal representing said movement of the heart plane exhibits a peak value in each cardiac cycle of the heart, and comprising analyzing said movement of the valve plane by identifying said peak value in one cardiac cycle.

56. A method as claimed in claim 47 wherein said electrical signal representing said movement of the heart plane exhibits a peak value in each cardiac cycle of the heart, and comprising analyzing said movement of the valve plane by forming an average of respective peak values from a plurality of cardiac cycles.

57. A method as claimed in claim 30 comprising storing said electrical signal representing said movement of the valve plane, together with a time at which said electrical signal was acquired, in a memory, and allowing access to, and subsequently analyzing, said electrical signal and said time stored in said memory.
